(19) United States
(12) Patent Application Publication
(10) Pub. No.: US 2023/0272327 A1
(43) Pub. Date: Aug. 31, 2023

(54) PERFUSION-ENABLED BIOREACTOR

(71) Applicant: University of Florida Research Foundation, Inc., Gainesville, FL (US)

(72) Inventors: Wallace Gregory SAWYER, Gainesville, FL (US); Ryan A. SMOLCHEK, Gainesville, FL (US); Juan Manuel URUEÑA VARGAS, Gainesville, FL (US); Jack E. FAMIGLIETTI, Gainesville, FL (US); Duy T. NGUYEN, Gainesville, FL (US)

(21) Appl. No.: 18/013,200

(22) PCT Filed: Jul. 2, 2021
(86) PCT No.: PCT/US2021/040264

§ 371 Date: Dec. 27, 2022

Related U.S. Application Data
(60) Provisional application No. 63/161,704, filed on Mar. 16, 2021.
(60) Provisional application No. 63/047,673, filed on Jul. 2, 2020.

(51) Int. Cl. C12M 1/00; C12M 1/12; C12M 1/34

(52) U.S. Cl. CPC C12M 29/10; C12M 25/04; C12M 41/40; C12M 23/34

(57) ABSTRACT

Provided herein are injection molded perfusion-enabled bioreactors and systems including said bioreactors. The bioreactor can include a lid, a frame comprising an an-ay of sample wells, a membrane adhered to a bottom of the frame beneath the array of sample wells, and a skirt adhered to a bottom of the frame, such that the membrane is located between the frame and the skirt. The skirt can include a plurality of channels corresponding to the sample wells. When the sample wells are filled with a 3D cell culture support matrix, a pressure above the 3D cell culture support matrix is atmospheric pressure and a pressure below the membrane is less than atmospheric, thereby perfusing a fluid along a vertical fluid flow path from the sample wells through the

### CROSS-REFERENCE TO RELATED APPLICATIONS

This application claims the benefit of and priority to U.S. Provisional Application Ser. No. 63/047,673, having the title “Clear Single-use Passive Perfusion Enabled 3D Bio-reactor Well Plate for Cell Culture and Experimentation”, filed on Jul. 2, 2020 and U.S. Provisional Application Ser. No. 63/161,704, having the title “PERFUSION-ENABLED BIOREACTOR”, filed on Mar. 16, 2021, the disclosures of each of which are incorporated herein by reference in their entireties.

### BACKGROUND

3D culture environments are needed for laboratory studies that are not entirely dependent on the use of animals or highly complex bioreactors. Animal care is often quite expensive and the results from animal studies are not always able to be properly translated into human biology. Furthermore, existing bioreactors for creating a 3D culture environment are often complicated, involving computer-controlled pumps and specially made parts, and may require highly trained personnel to operate them. In short, both existing pathways into 3D culture tend to have a high barrier to entry in terms of cost and skill.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 is a diagram illustrating an exploded view of a perfusion-enabled bioreactor in accordance with embodiments of the present disclosure.

FIGS. 2A-2B show the bioreactor 100 from FIG. 1 in a closed, assembled position in accordance with embodiments of the present disclosure. FIG. 2A is a perspective view, FIG. 2B is a bottom view.

FIG. 3 is a diagram illustrating a section view of the perfusion-enabled bioreactor shown in FIG. 2, in accordance with embodiments of the present disclosure.

FIGS. 4A-4F provide diagrams illustrating various views of a perfusion-enabled bioreactor in accordance with embodiments of the present disclosure. FIG. 4A is a multi-section view in which the bioreactor is divided into quadrants to show the interior at various depths; FIG. 4B is a top view of the bioreactor; FIGS. 4C and 4D are end and side views, respectively; FIGS. 4E and 4F are cross-section views from the side, cut along the sections indicated in FIG. 4D.

FIGS. 5A-5F provide diagrams of the frame from various angles. FIG. 5A shows a top perspective view, FIG. 5B shows a bottom perspective view; FIG. 5C shows a bottom view; FIG. 5D shows a top view; FIG. 5E shows a side view; and FIG. 5F shows an end view.

FIGS. 6A-6F provide diagrams of the base from various angles. FIG. 6A shows a top perspective view, FIG. 6B shows a bottom perspective view; FIG. 6C shows a top view; FIG. 6D shows a bottom view; FIG. 6E shows a side view; and FIG. 6F shows an end view.

FIGS. 7A-7C provide diagrams of the lid from various angles. FIG. 7A shows a bottom perspective view, FIG. 7B shows a top perspective view; and FIG. 7C shows a bottom view (underside of the lid).

FIGS. 8A-8E provide diagrams of the skirt from various angles. FIG. 8A shows a top perspective view, FIG. 8B shows a bottom perspective view; FIG. 8C shows a top view; FIG. 8D shows a bottom view; and FIG. 8E shows a side view.

FIGS. 9A and 9B provide an exploded view and an assembled view, respectively, of a bioreactor molded from transparent materials in accordance with embodiments of the present disclosure.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of biology, material science, mechanical engineering, and the like, which are within the skill of the art.

The following examples are put forth so as to provide those of ordinary skill in the art with a complete disclosure and description of how to perform the methods and use the materials and devices disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

As used herein, the following terms have the meanings ascribed to them unless specified otherwise. In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

### General Discussion

In accordance with the purpose(s) of the present disclosure, as embodied and broadly described herein, embodiments of the present disclosure, in some aspects, relate to perfusion-enabled bioreactors, systems including perfusion-enabled bioreactors, and methods for 3D cell culture using perfusion-enabled bioreactors.

The perfusion-enabled bioreactors described herein are designed as part of a system for the efficient and effective culture of cells or other biological materials in three dimensions (3D). The perfusion-enabled bioreactors described herein can be used in conjunction with a gel-like support matrix in which the cells or biological material has been seeded. Using a low-pressure gradient, the system can drive flow of a liquid culture medium across the cells and perfuse the liquid through the support matrix and an underlying microporous membrane and into a collection reservoir. Constant flow of fresh culture medium over the cultures allows for maintaining viability in the cultures, as it is this flow that washes away metabolic wastes and brings in fresh nutrients and other factors from the fresh media. Advantageously, in the bioreactors and systems described herein, the perfusion flow can run consistently for long periods of time before the liquid media in the top of the bioreactor (also referred to as a plate or perfusion plate) is replenished. In some embodiments, the perfusion flow can run consistently for about 24 hours before the liquid is replenished. The flow rate can also be tuned by adjusting one or more of the membrane porosity and gel makeup. In some embodiments, the perfusion flow can run consistently for longer than 24 hours before the liquid is replenished.

Advantageously, the perfusion-enabled bioreactors and systems described herein allow for constant perfusion flows over the cultures without complicated pumps/machines or modification to existing incubators used in traditional culture.

The perfusion-enabled bioreactors and systems described herein can be used for a wide array of 3D culture experiments, some of which may not be envisioned yet. Some applications include the study the effects of drugs on cell cultures including cancers and other diseases, and cells-cells interaction (e.g. immune cells—tumor interaction) in 3D, in vitro and in silico cell motility, and multi organoids co-culture studies.

The present disclosure includes a perfusion-enabled bioreactor that includes a lid, a frame including a sample well or an array of sample wells and a base. The base can include at least one reservoir. The perfusion-enabled bioreactor can include a membrane located beneath the frame. The membrane can be adhered to a bottom of the sample well(s). The base can be wider than the frame and lid. A locking mechanism can seal the frame to the base. At least the base and the frame can be injection molded. The lid may also be injection molded.

In some embodiments, the perfusion-enabled bioreactor can include a skirt adhered to a bottom of the frame, where the membrane is sandwiched between the frame and the skirt. The skirt can have a channel or channels, each channel correlating to a sample well and leading to the reservoir or reservoirs in the base. Advantageously, the skirt can guide droplets forming from each well into each of the quadrants and prevent wicking across to other wells. The skirt can also reinforce membrane adherence to the frame. The skirt can also be injection molded.

In some embodiments, the perfusion-enabled bioreactor can be disposable (e.g. single-use or used as a consumable). Advantageously, a disposable perfusion-enabled bioreactor allows for sterilization and packaging prior to use in a study and minimal clean-up afterward without need to consider re-sterilization for reuse. Furthermore, the easily-reproduced complex geometries that can be made with injection molding means the device can incorporate a wide array of features while still being mass producible in a short time. These features can include such as visual feedback using graduations in the collection reservoir, pressure port geometry for connecting the negative pressure tube, glue relief channels for attaching the membrane without clogging wells, and the elastomer ring locking mechanisms which ensure proper sealing by slightly clamping down on the assembly.

The frame can include an array of sample wells. The frame can include recesses where the wells or groups of wells are in the bottom of the recess. In a particular embodiment, the frame can include 24 individual wells, which can be split into four quadrants, each quadrant having a recess with six wells. There can be a corresponding divider in the base. In some embodiments, the base can include a plurality of separate reservoirs. The base can be divided into quadrants such that there are four reservoirs (e.g. collection wells), one in each quadrant. The skirt is adhered to the bottom of the frame, so that the microporous membrane is sandwiched between the skirt and the frame. The membrane can be heat sealed or glued to the bottom of the wells in the frame and the skirt is adhered to the frame (e.g. by an adhesive). The skirt provides a direct channel from each of the individual wells down into the collection reservoir for the corresponding quadrant of the plate. Accordingly, the base is divided into multiple reservoirs, and each of the channels directs fluid from a sample well into a specific reservoir. Advantageously, this arrangement ensures that the effluent from each well falls into the appropriate reservoir without wicking over into other regions where the experimental conditions may be different.

In some embodiments, the base can include a single reservoir such that all the effluent from all wells collects in one large reservoir. In such an embodiment, the skirt can be omitted.

In yet other embodiments, different array patterns or numbers of wells can be envisioned by one of ordinary skill in the art. For example, the frame can have such as 24 wells divided into 6 groups, 36 wells divided into 6 groups, 12 wells divided into 4 groups, etc. Each group can be in a recess. The base can have corresponding dividers to create a reservoir for each group of wells in the frame.

In some embodiments, depressed fill ports can be included in each of the well groups to be used when filling the plate to prevent disruption of the gel in each well. Advantageously, the fill port does not allow direct jets of fluid to push gel around or disrupt the seeded cells. The fill ports can be hemispherical depressions. The fill ports can accommodate such as an 18-gauge needle and a syringe. The recess above the wells can be filled by inserting the needle into the fill port and slowly depressing the plunger on the syringe. In this manner, liquid (e.g. liquid culture media) can be added to the recess above the gel in each of the wells. The fill ports allow for the turbulence from a liquid stream from the needle to be directed upward and not into or around the surrounding wells full of gel. Without the fill ports, the addition of liquid during the initial experimental setup or during liquid replenishment could disrupt the gel.

In some embodiments, the frame contains a pressure port. In some embodiments, there can be a pressure port for each reservoir. The pressure port can serve as both a media collection port and a port for tubing. The pressure port is designed so that a tube can be press fit into the pressure port, the press fitting action sealing the tube. Advantageously, any mechanism to actuate a low pressure could be used in conjunction with this tube (e.g. a bulb, pump, syringe, etc). The value of the pressure used to drive the perfusion flow is variable and can be changed based on experimental needs.

Advantageously, the reservoirs in the base can be accessed individually via such as a syringe needle through the port used to actuate the pressure change. The effluent media can be collected individually from each of the reservoirs without having to disassemble the plate. The base dividers can be arranged such that liquid from each of the reservoirs can be accessed through a single port.

In some embodiments, the frame can include an interference fit pressure port, which allows for any vacuum mechanism with compatible tubing diameter to be used.

In some embodiments, the pressure port can be located in the base.

The lid can sit atop the plate to cover the wells from airborne contaminants that could disturb an experiment. The lid can include a 1-2 mm downward facing air gap to allow gas exchange while preventing contaminants from falling into the wells. The lid is designed to sit flush with the frame. As mentioned above, the widest and largest part of the plate is the base. In conjunction with the flush lid, there is a reduced risk of a user accidentally picking up the lid rather than the entire unit, thus exposing the plate to contaminants.

In some embodiments, the lid can include protrusions on the underside to center the lid on the frame and provide a sufficient gap for gas exchange. In some embodiments, the protrusions correspond to gaps dividing groups of sample wells in the frame. In some embodiments, an air filter can be installed in the air gaps to further reduce infiltration of contaminants. The lid may also include a gas port to flow desired gasses directly onto media in the bioreactor.

One or more of the lid, frame, base, and skirt can be injection molded. The injection molded parts can be made out of any injection moldable plastic or polymer that is stable up to physiological temperatures (e.g. about 37° C.). Suitable materials include but are not limited to polystyrene, nylon, acrylic, polycarbonate, polyoxymethylene, acrylonitrile butadiene styrene (ABS), polypropylene, polyethylene, and the like.

In some embodiments, the injection molded parts can be optically transparent, which allows for monitoring of levels of liquid media in the top and bottom of the plate without the need to disassemble it. Advantageously, the use of thin-walled transparent plastic parts without fasteners increases the overall allowable volume of liquid culture media without increasing the footprint of the assembly and gives useful visual feedback on perfusion volumes.

The frame can be secured onto the base with a locking mechanism. In some embodiments, the frame is pressed onto the base via an elastic band (e.g. an o-ring or gasket) on each end of the plate, the band functioning as the locking mechanism. The assembled bioreactor can include grooves on each end to prevent the elastic bands from sliding out of position. The base and the frame can be designed to mate to form a seal (e.g. a labyrinth seal or a compression seal). For example, the base can include a channel around the upper edge which is filled with a soft elastomer material (e.g. a gasket or a silicone-based material). The channel can receive a protruding ridge on the bottom of the frame. The clamping force applied by the elastic bands around each end of the assembled plate can force the frame down onto the base and thus push the protruding ridge on the frame into the elastomer-filled sealing channel on the base. The frame and bottom can form a seal along this edge due to the compression of the elastomer in the sealing channel. In other embodiments, the sealing channel can be in the frame and the protrusions can be in the base. The sealing creates an air space in the assembled plate that is only accessible through the pressure port or through the microporous membrane at the bottom of each of the wells. In some embodiments, the channel can be filled with elastomer material that is cured to form the seal. In other embodiments, a gasket, o-ring, or other elastic seal can be fitted to the channel. Alternatively, the locking mechanism can be clamps, clips, latches, snap closures, or other suitable closure sufficient to apply force such that the base and the frame are sealably mated.

Embodiments of the present disclosure include a system including a perfusion-enabled bioreactor as above, wherein the base of the well plate is configured to be sealably mated with the frame so that when mated the sample wells are in fluidic communication with the reservoirs via the channels of the skirt. When the sample wells are filled with a 3D cell culture support matrix, the system is configured so that a pressure above the 3D cell culture support matrix is atmospheric pressure and a pressure below the membrane is a negative pressure (e.g. a pressure which is less than the atmospheric pressure). The 3D cell culture support matrix contains seeded cells and sits atop the microporous membrane. A liquid cell culture media is flowed on top of the 3D cell culture support matrix. The negative pressure perfuses the liquid cell culture media along a vertical fluid flow path from the sample wells through the 3D cell culture support matrix, through the membrane, and along the channels to the reservoir. The perfused fluid can then be collected from the reservoir though one or more ports, such as with a syringe.

The microporous membrane can be comprised of such as polycarbonate, cellulose, nylon, PEEK, polypropylene, or combinations thereof.

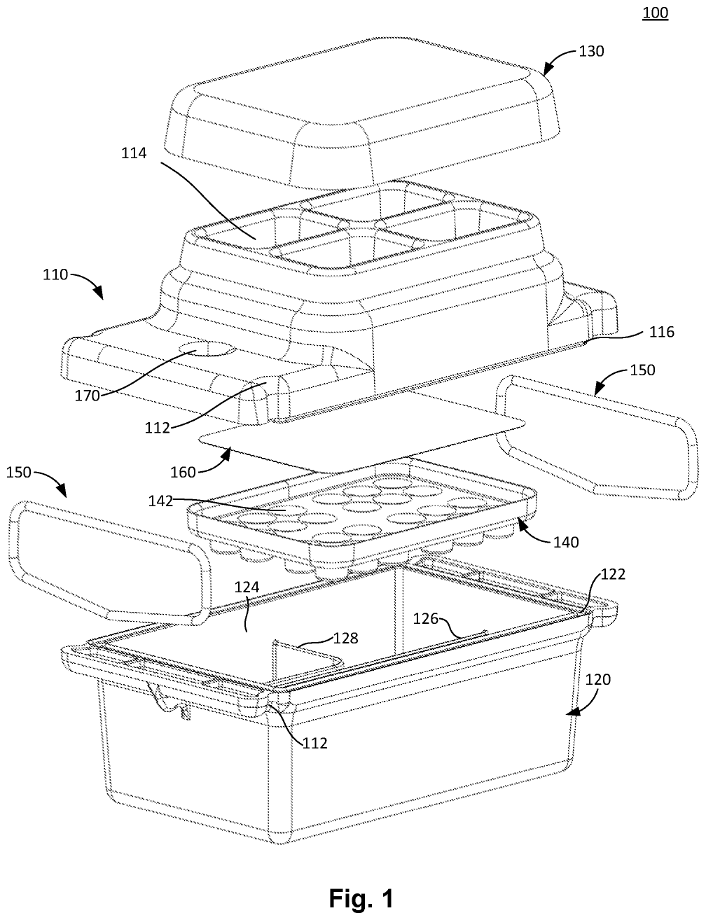

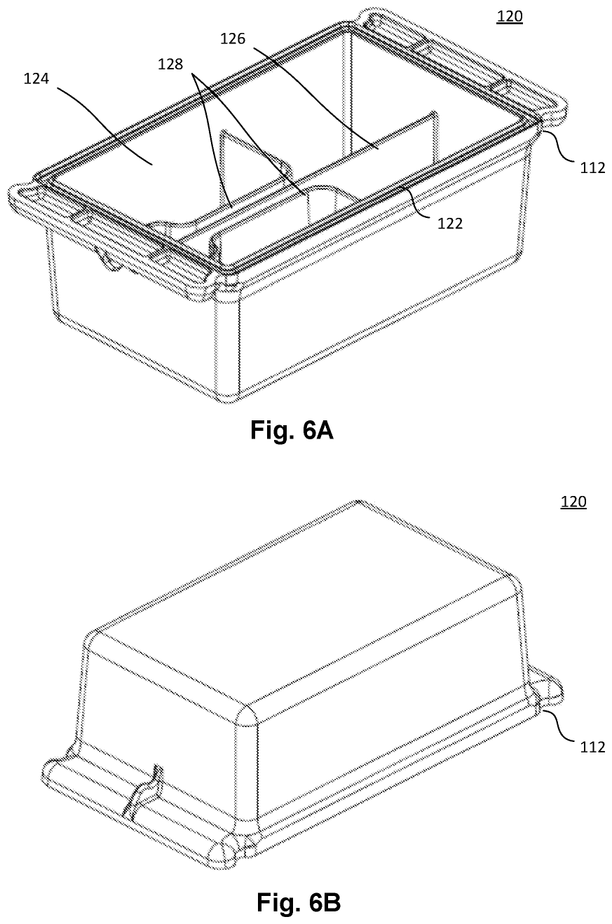

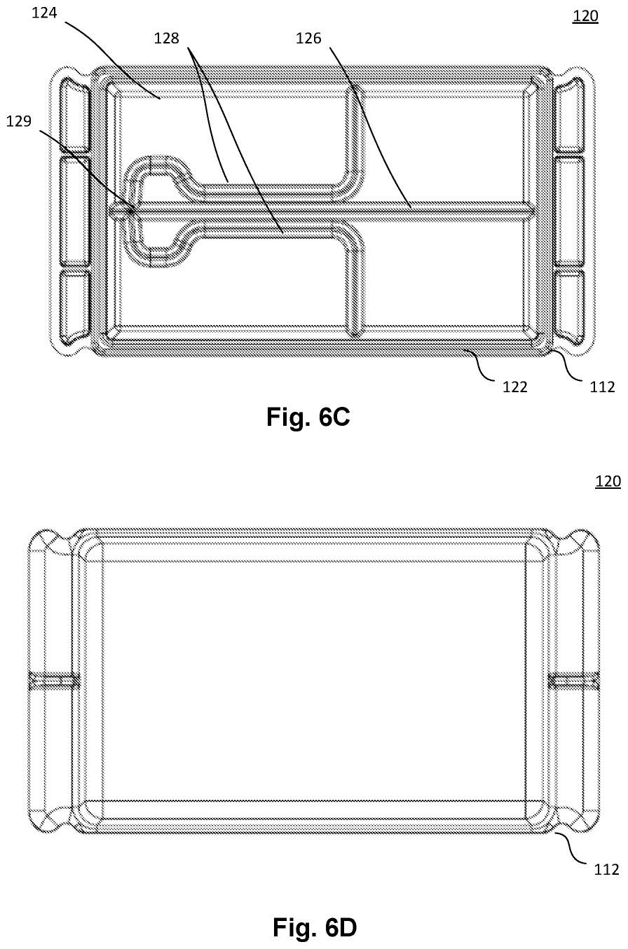

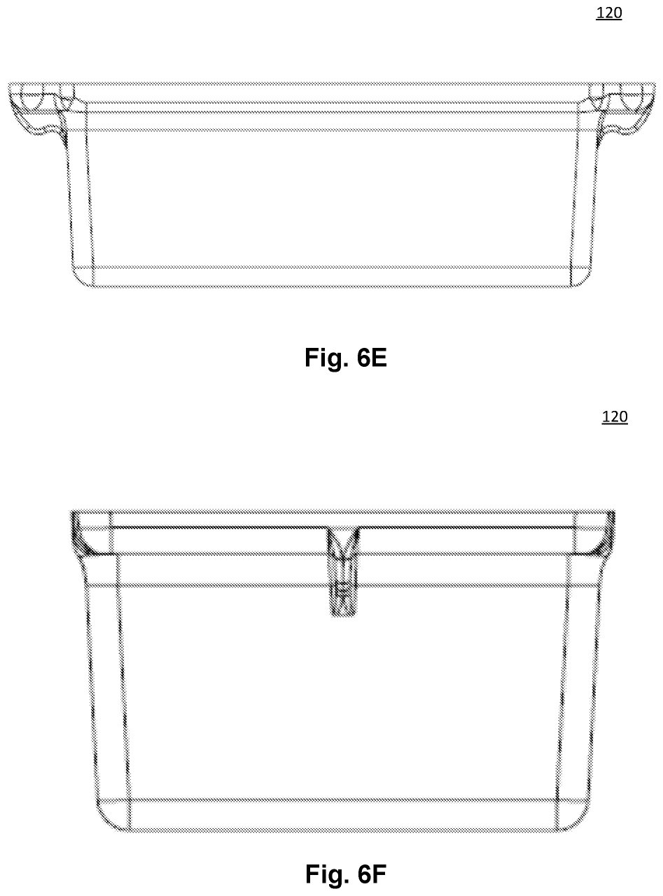

Turning now to the figures, FIG. 1 provides an exploded view of an example perfusion-enabled bioreactor 100. This example includes a frame 110, a portion of which is divided into four quadrants. Each quadrant having a recess 114. Membrane 160 seals to the bottom of the frame 110. Skirt 140 adheres to the quadrant portion on the bottom of the frame 110, sandwiching the membrane 160. Skirt 140 contains channels 142 that correspond to sample wells 180 located in the frame 110 (sample wells 180 are not visible in this view). The frame 110 has protruding ridges 116 which seat into a sealing channel 122 of base 120. The sealing channel 122 includes an elastomeric material 123 (not shown). Locking mechanism 150 is a gasket that compresses the frame 110 and base 120 together to form a seal. The frame 110 and base 120 have grooves 112 to hold the locking mechanism 150 in place. Lid 130 sits atop the frame with a small amount of clearance inside (e.g. about 1 mm-2 mm) to allow for gas exchange. Reservoir 124 is formed in base 120 by a divider 126 and one or more curvy dividers 128, described in further detail in FIGS. 6A-6F.

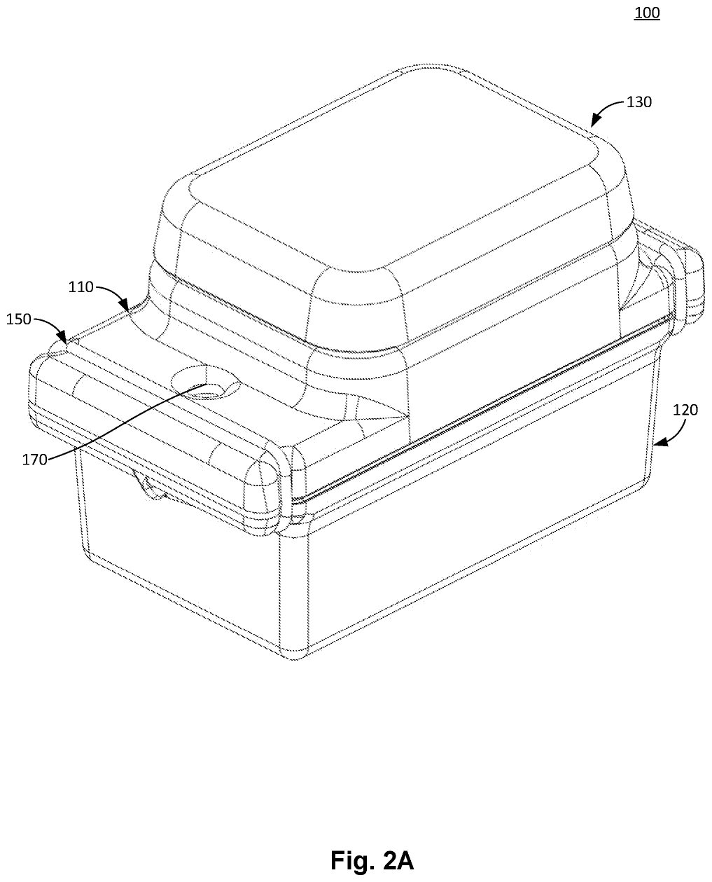

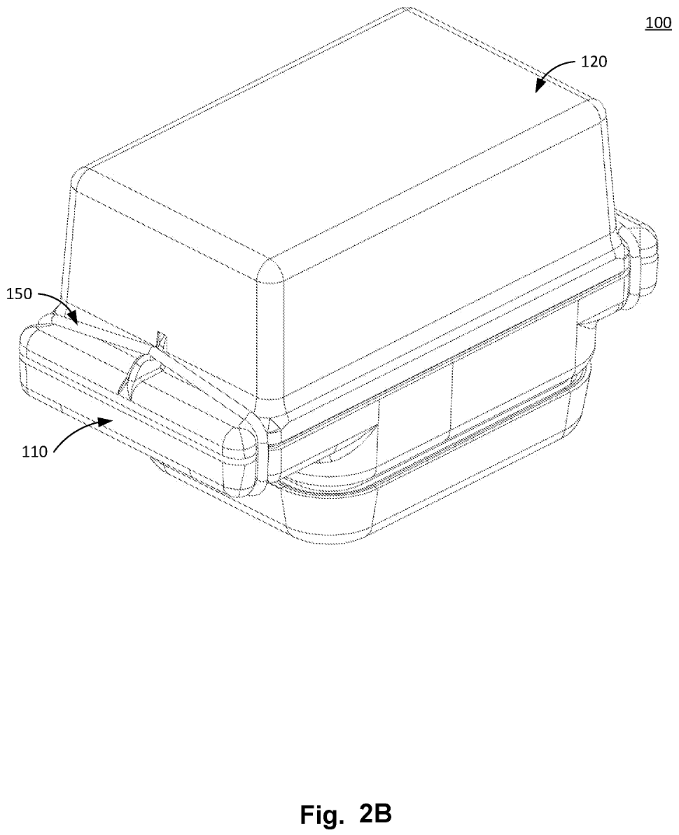

FIG. 2A shows a perspective view of the bioreactor 100 from FIG. 1 in a closed, assembled position. Pressure port 170 is shown on frame 110. FIG. 2B is a perspective view of the unit upside down to show the base 120.

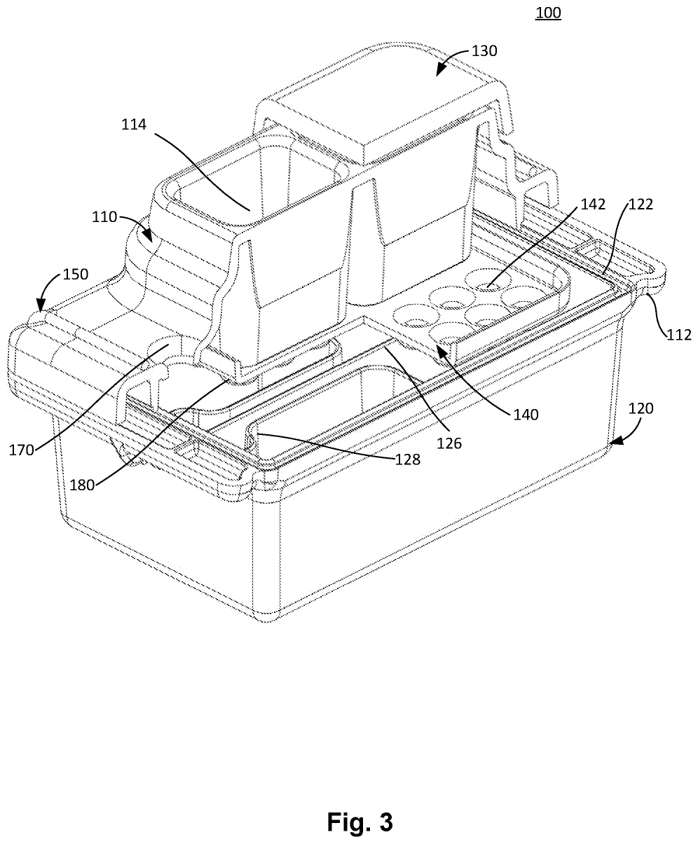

FIG. 3 provides a section view of the bioreactor 100 shown in FIGS. 1-2E. The membrane 160 is not shown in this view. As can be seen from corresponding channels 142 in the skirt 140, each of the quadrants in the frame 110 includes 6 sample wells 180. The grouped sample wells 180 flow through the channels into the separate reservoirs 124 formed in the base 120.

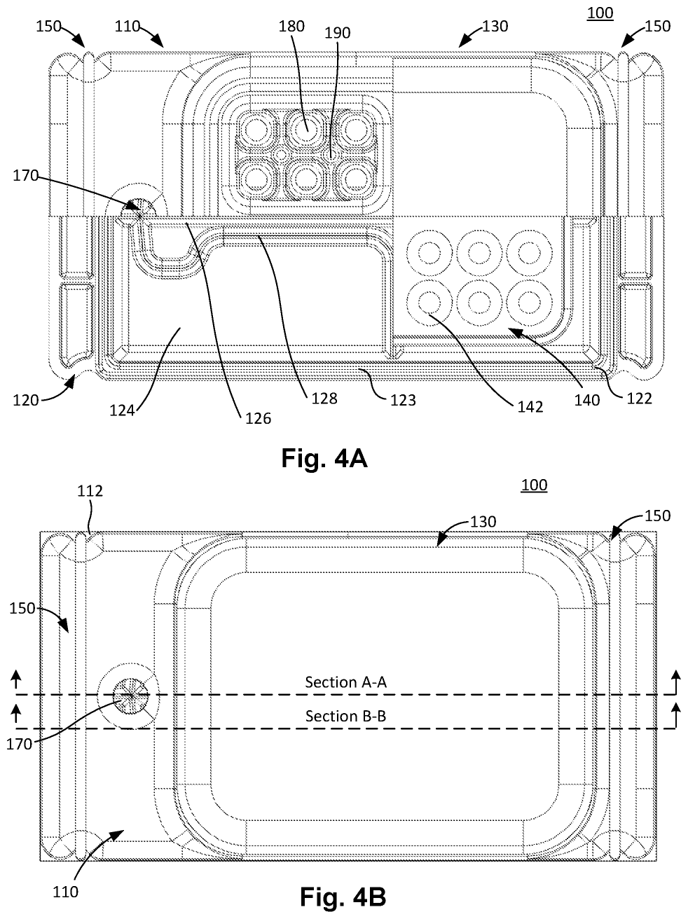

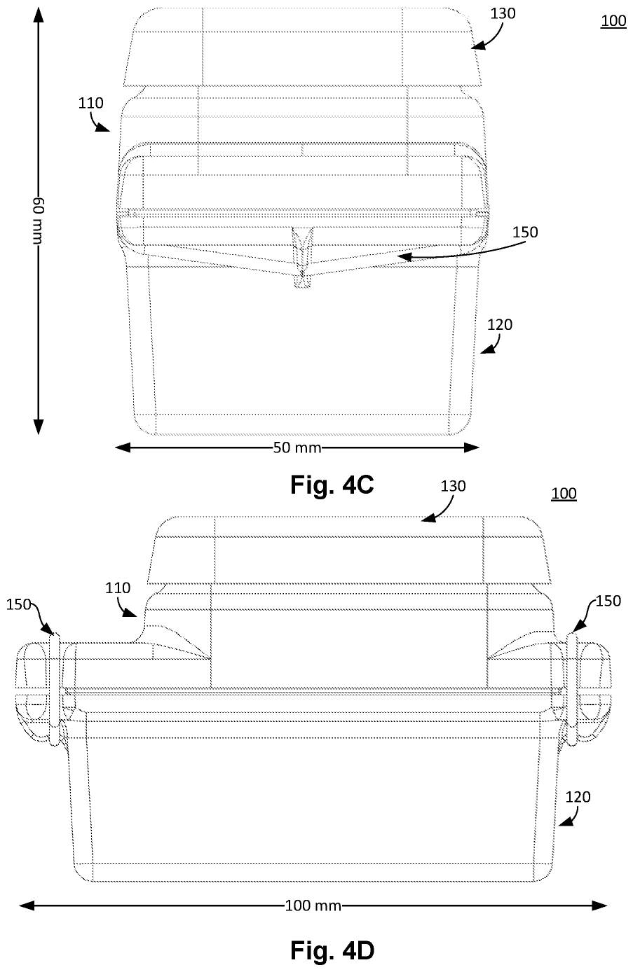

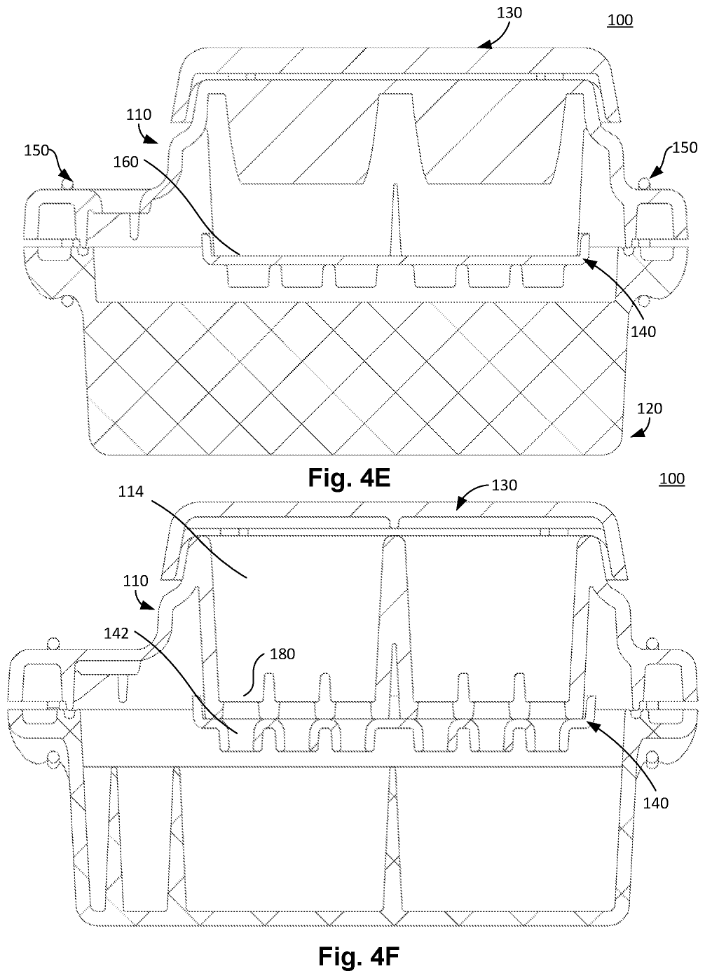

FIGS. 4A-4F provide diagrams illustrating various views of another example of a perfusion-enabled bioreactor. FIG. 4A is a multi-section view from the top in which the bioreactor is divided into quadrants to show the interior at various depths. The top left shows the frame 110, with the sample wells 180 grouped together. Sample wells 180 and fill ports 190 can be seen in this section. The top right shows the lid 130. The bottom left shows the reservoir 124 in the base 120 that corresponds to the shape of the group of sample wells 180 that seats in from the frame 110 above. The bottom right quadrant shows the skirt having 6 channels to correspond to the 6 sample wells in the frame. As can be appreciated from this view, the reservoir 124 in the base 120 (bottom left quadrant of the figure) can be accessed through the pressure port 170 in the frame 110 (top left quadrant). The reservoir 124 is separated into quadrants by dividers 126 and 128 (see descriptions of FIGS. 6A-6F for a detailed description). FIG. 4B is a top view of the bioreactor. FIGS. 4C and 4D are end and side views of the assembled bioreactor, respectively. Example dimensions of the bioreactor are provided. In this example, the entire unit has a height of about 60 mm, a width of about 50 mm, and a length of about 100 mm. The unit can be scaled as can be envisioned by one of ordinary skill in the art. FIGS. 4E and 4F are cross-section views from the side, cut along the sections A-A and B-B, respectively, as indicated in FIG. 4F. In FIG. 4E, the membrane 160 is indicated seated between the sample wells 180 and the skirt 140. FIG. 4F illustrates the mating of sample wells 180 and skirt channels 142.

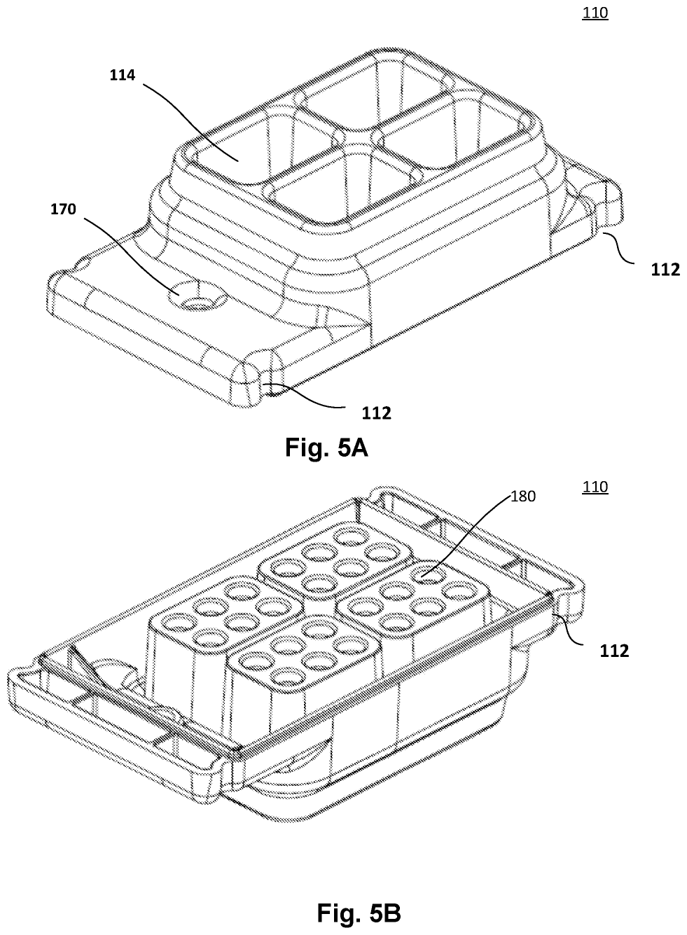

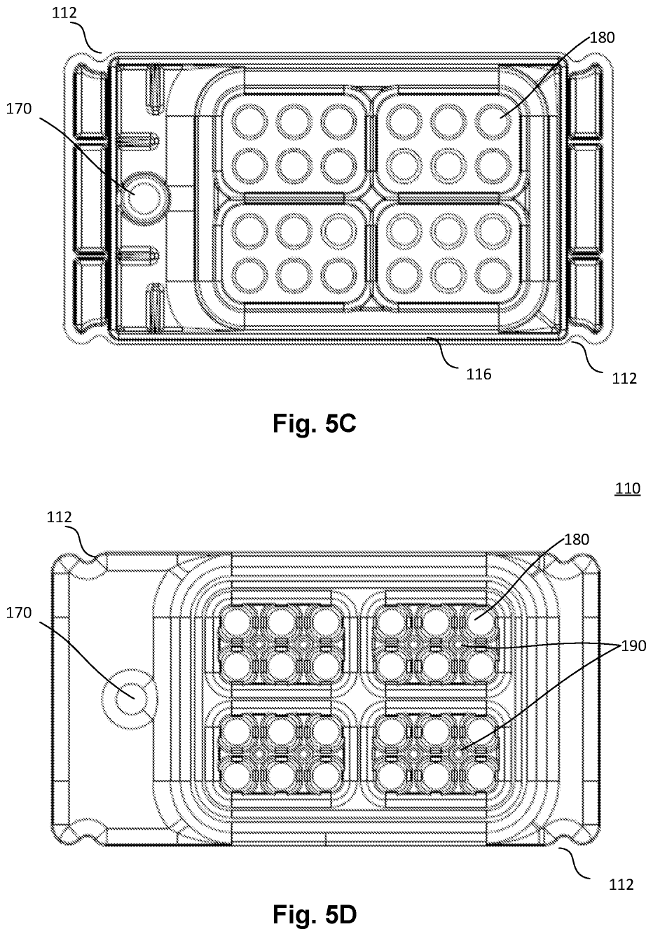

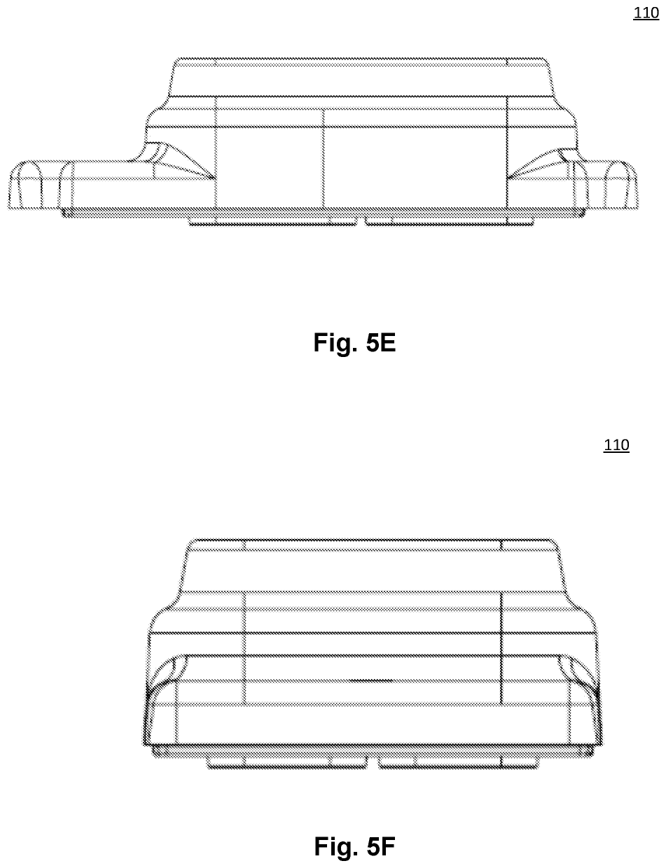

FIGS. 5A-5F provide diagrams of an embodiment of the frame 110 from various angles. FIG. 5A shows a top perspective view in which the frame has been divided into quadrants of sample wells 180. Recesses 114 and pressure port 170 are seen. FIG. 5B shows a bottom perspective view in which the 6 sample wells per quadrant recess 114 can be seen. FIG. 5C shows a bottom view; FIG. 5D shows a top view in which fill ports 190 can be seen; FIG. 5E shows a side view; and FIG. 5F shows an end view.

FIGS. 6A-6F provide diagrams of the base 120 from various angles. This base is configured to receive a frame 110 divided into quadrants as shown in FIGS. 5A-5F. FIG. 6A shows a top perspective view, FIG. 6B shows a bottom perspective view; FIG. 6C shows a top view; FIG. 6D shows a bottom view; FIG. 6E shows a side view; and FIG. 6F shows an end view. As can be appreciated by the figures, especially FIGS. 6A and 6C, the base is divided into four reservoirs 124 by dividers. In the shown embodiment, there is one long divider 126 across the length of the plate. There are then two mirrored “curvy” dividers 128 on either side of the long divider 126. These curvy dividers 128 act to extend the reservoirs on the side of the plate furthest from the pressure port (two reservoirs on right side of FIG. 6C). At the end of the divider 128 having the curved portion, there is a point at which each of the dividers meet, forming a plus shape (+). This point 129 is located directly beneath the pressure port 170 (also referred to as a media collection port). Through the pressure port 170, each reservoir can be reached with a needle and individually collected without removing the frame 110 from the base 120.

The liquid media perfuses through the gel layer in each well, then falls by way of the skirt 140 into its collection reservoir 124. When the reservoirs 124 are full, which can be seen through the clear base, one needs to empty the reservoirs so that the perfusion can be continued without overfilling the base 120. This media removal can be done without separating the frame 110 from the base 120 by drawing up the media in the reservoirs 124 through the media collection port (pressure port 170). The plus (+) shaped area 129 of the dividers in the base 120 allows for media from each reservoir 124 to be accessed with a needle through the media collection (pressure) port 170.

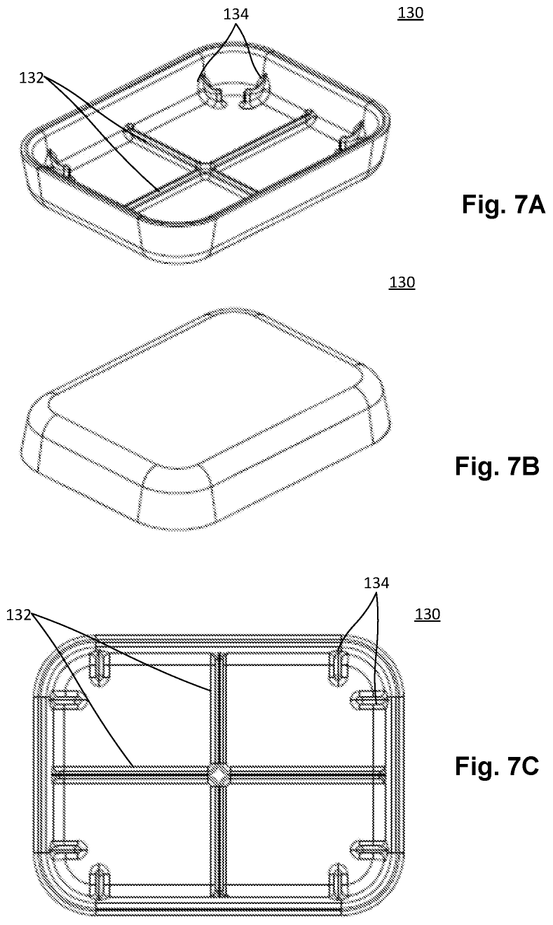

FIGS. 7A-7C provide diagrams of the lid 130 from various angles. FIG. 7A shows a bottom perspective view (underside of the lid). The lid 130 includes dividers 132 that correspond to the recesses 114 in the frame 110 and/or provide structural support to the lid 130. The curved corner supports 134 in the corners of the lid sit on the frame 110. The curved corner supports 134 enable the lid 130 to be automatically centered on the frame 110. This creates a consistent air gap between the edge of the lid 130 and the top of the frame 110 all the way around the bioreactor 100. FIG. 7B shows a top perspective view; and FIG. 7C shows a bottom view (underside of the lid). As shown in FIG. 7C, in some embodiments, the corner supports 134 may be straight.

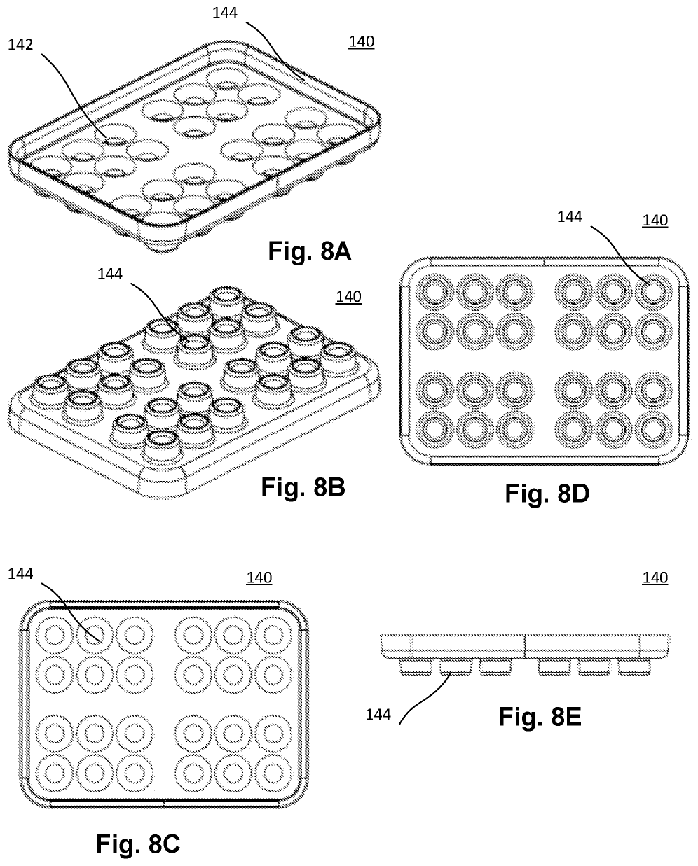

FIGS. 8A-8E provide diagrams of the skirt 140 from various angles. FIG. 8A shows a top perspective view, FIG. 8B shows a bottom perspective view; FIG. 8C shows a top view; FIG. 8D shows a bottom view; and FIG. 8E shows a side view. The skirt 140 includes a lip 144 to provide a seat for the frame 110. In this example, the channels 142 correspond to a 24-well array such as the one shown in FIGS. 5A-5F.

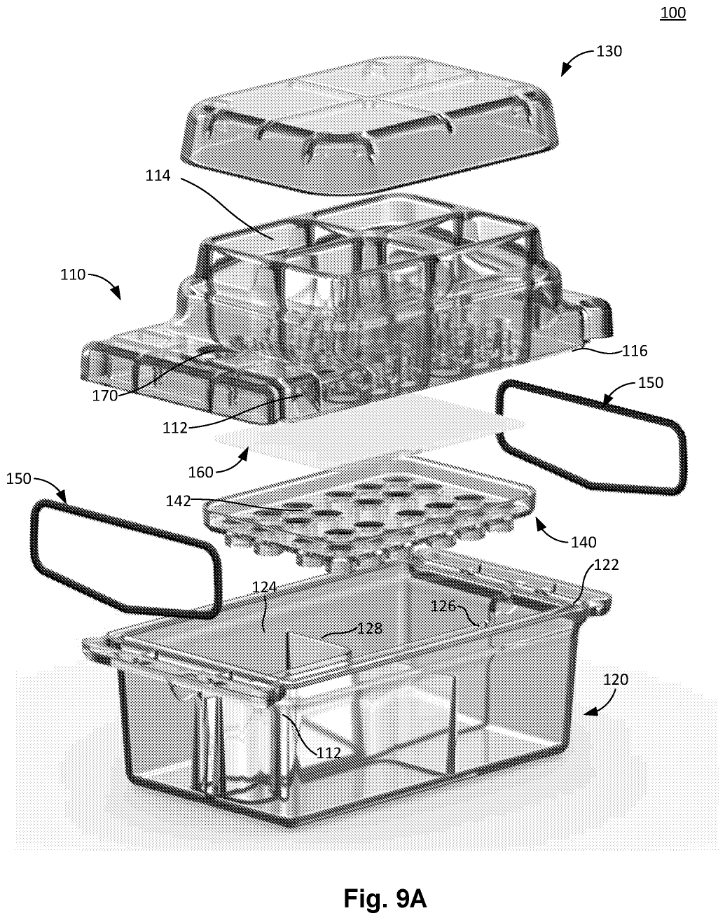

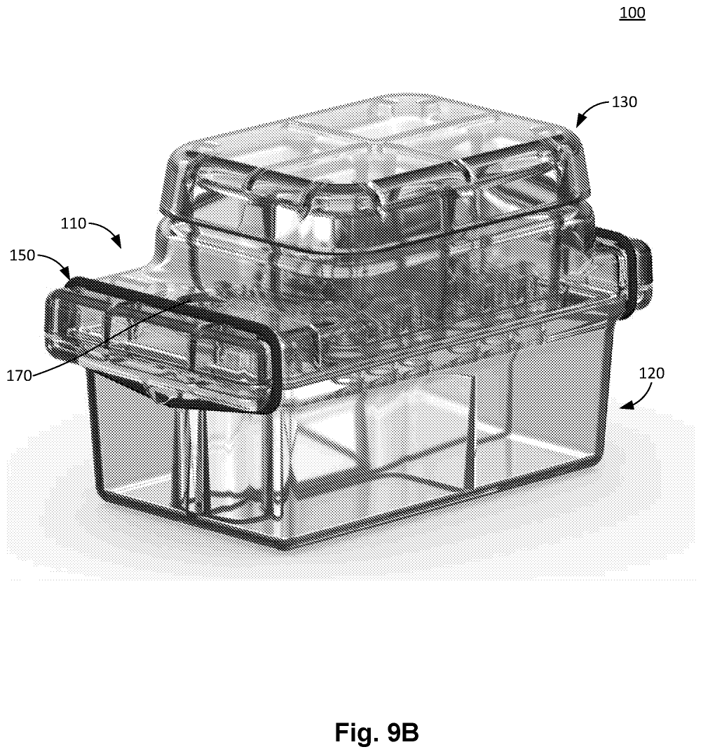

FIGS. 9A and 9B provide an exploded view and an assembled view, respectively, of a bioreactor 100 molded from transparent materials.

### ASPECTS OF THE DISCLOSURE

The present disclosure will be better understood upon reading the following numbered aspects, which should not be confused with the claims. Any of the numbered aspects below can, in some instances, be combined with aspects described elsewhere in this disclosure and such combinations are intended to form part of the disclosure.

Aspect 1. A perfusion-enabled bioreactor, comprising a lid; a frame comprising at least one sample well in a recess and at least one fill port; a membrane located beneath the frame, wherein the membrane is adhered to a bottom of the at least one sample well; a base comprising at least one reservoir, wherein the base is the wider than the frame and lid; and a locking mechanism that seals the frame to the base; wherein at least the frame and base are injection molded.

Aspect 2. The perfusion-enabled bioreactor according to aspect 1, further comprising a skirt adhered to a bottom of the frame, such that the membrane is located between the frame and the skirt, the skirt comprising a channel leading from each sample well to the reservoir.

Aspect 3. The perfusion-enabled bioreactor according to any preceding aspect, wherein the frame comprises an array of sample wells, wherein the base is divided into multiple reservoirs, and wherein each of the channels directs fluid from a sample well into a specific reservoir.

Aspect 4. The perfusion-enabled bioreactor according to aspect 3, wherein the frame contains a media collection port from which fluid can be collected from each reservoir.

Aspect 5. The perfusion-enabled bioreactor according to any preceding aspect, wherein the locking mechanism comprises a pair of gaskets that clamp the frame to the base at each end of the bioreactor.

Aspect 6. The perfusion-enabled bioreactor according to any preceding aspect, wherein the base includes a channel around a top edge to receive a bottom edge of the frame such that the base and the frame are sealably mated.

Aspect 7. The perfusion-enabled bioreactor according to any preceding aspect, wherein a bottom of the lid sits flush with the frame, and wherein the lid comprises a raised center portion providing about 1 to 2 mm clearance from the frame.

Aspect 8. The perfusion-enabled bioreactor according to any preceding aspect, wherein the membrane is adhered to the bottom of the plurality of wells by heat sealing.

Aspect 9. The perfusion-enabled bioreactor according to any preceding claim, wherein the membrane is comprised of polycarbonate, cellulose, nylon, PEEK, polypropylene, or combinations thereof.

Aspect 10. The perfusion-enabled bioreactor according to any preceding aspect, wherein one or more of the lid, the frame, the base, and the skirt are transparent.

Aspect 11. The perfusion-enabled bioreactor according to any preceding aspect, wherein the frame comprises a plurality of recesses, wherein each recess contains a plurality of sample wells and at least one fill port.

Aspect 12. A system for 3D cell culture, comprising an injection molded perfusion-enabled bioreactor, the perfusion-enabled bioreactor comprising: a lid; a frame comprising at least one recess having an array of sample wells and at least one fill port; a membrane adhered to a bottom of the frame beneath the array of sample wells; a skirt adhered to a bottom of the frame, such that the membrane is located between the frame and the skirt, the skirt comprising a plurality of channels; a base comprising at least one reservoir, wherein the base is the wider than the frame and lid; and a locking mechanism that seals the frame to the base; wherein the base of the well plate is configured to be sealably mated with the frame so that when mated the sample wells are in fluidic communication with the reservoirs via the channels of the skirt; wherein the frame comprises a media collection port through which media can be collected from each reservoir in the base; and wherein when the sample wells are filled with a 3D cell culture support matrix, the system is configured so that a pressure above the 3D cell culture support matrix is atmospheric pressure and a pressure below the membrane is a negative pressure, wherein the negative pressure perfuses a fluid along a vertical fluid flow path from the sample wells through the 3D cell culture support matrix, through the membrane, and along the channels to the reservoir.

Aspect 13. The system of aspect 12, wherein the port is configured to allow a sample of the perfused fluid to be collected from the reservoir with a syringe.

Aspect 14. The system of aspect 12, wherein the negative pressure is drawn by a tube connected to the port.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, “about 0” can refer to 0, 0.001, 0.01, or 0.1. In an embodiment, the term “about” can include traditional rounding according to significant figures of the numerical value. In addition, the phrase “about ‘x’ to ‘y’” includes “about ‘x’ to about ‘y’”.

It should be emphasized that the above-described embodiments of the present disclosure are merely possible examples of implementations, and are set forth only for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure.

1. A perfusion-enabled bioreactor, comprising:
a lid;
a frame comprising at least one sample well in a recess and at least one fill port;
a membrane located beneath the frame, wherein the membrane is adhered to a bottom of the at least one sample well;
a base comprising at least one reservoir, wherein the base is wider than the frame and lid; and
a locking mechanism that seals the frame to the base;
wherein at least the frame and base are injection molded.

2. The perfusion-enabled bioreactor according to claim 1, further comprising a skirt adhered to a bottom of the frame, such that the membrane is located between the frame and the skirt, the skirt comprising a channel leading from each sample well to the reservoir.

3. The perfusion-enabled bioreactor according to claim 2, wherein the frame comprises an array of sample wells, wherein the base is divided into multiple reservoirs, and wherein each of the channels directs fluid from a sample well into a specific reservoir.

4. The perfusion-enabled bioreactor according to claim 3, wherein the frame contains a media collection port from which fluid can be collected from each reservoir.

5. The perfusion-enabled bioreactor according to claim 1, wherein the locking mechanism comprises a pair of gaskets that clamp the frame to the base at each end of the bioreactor.

6. The perfusion-enabled bioreactor according to claim 1, wherein the base includes a channel around a top edge to receive a bottom edge of the frame such that the base and the frame are sealably mated.

7. The perfusion-enabled bioreactor according to claim 1, wherein a bottom of the lid sits flush with the frame, and wherein the lid comprises a raised center portion providing about 1 to 2 mm clearance from the frame.

8. The perfusion-enabled bioreactor according to claim 1, wherein the membrane is adhered to the bottom of the plurality of wells by heat sealing.

9. The perfusion-enabled bioreactor according to claim 1, wherein the membrane is comprised of polycarbonate, cellulose, nylon, PEEK, polypropylene, or combinations thereof.

10. The perfusion-enabled bioreactor according to claim 1, wherein one or more of the lid, the frame, the base, and the skirt are transparent.

11. The perfusion-enabled bioreactor according to claim 1, wherein the frame comprises a plurality of recesses, wherein each recess contains a plurality of sample wells and at least one fill port.

12. A system for 3D cell culture, comprising:
an injection molded perfusion-enabled bioreactor, the perfusion-enabled bioreactor comprising:
a lid;
a frame comprising at least one recess having an array of sample wells and at least one fill port;
a membrane adhered to a bottom of the frame beneath the array of sample wells;
a skirt adhered to a bottom of the frame, such that the membrane is located between the frame and the skirt, the skirt comprising a plurality of channels;
a base comprising at least one reservoir, wherein the base is wider than the frame and lid; and
a locking mechanism that seals the frame to the base;
wherein the base of the well plate is configured to be sealably mated with the frame so that when mated the sample wells are in fluidic communication with the reservoirs via the channels of the skirt;
wherein the frame comprises a media collection port through which media can be collected from each reservoir in the base; and
wherein when the sample wells are filled with a 3D cell culture support matrix, the system is configured so that a pressure above the 3D cell culture support matrix is atmospheric pressure and a pressure below the membrane is a negative pressure, wherein the negative pressure perfuses a fluid along a vertical fluid flow path from the sample wells through the 3D cell culture support matrix, through the membrane, and along the channels to the reservoir.

13. The system of claim 12, wherein the media collection port is configured to allow a sample of the perfused fluid to be collected from the reservoir with a syringe.

14. The system of claim 12, wherein the negative pressure is drawn by a tube connected to the media collection port.
